(19) United States
(12) Patent Application Publication
(10) Pub. No.: US 2011/0305778 A1
(43) Pub. Date: Dec. 15, 2011

(54) Chemically Stabilized Anti-Dandruff Compositions For Use In Personal Care Compositions

(76) Inventors: Marco Caggioni, Cincinnati, OH (US); Douglas Allan Royce, Sunman, IN (US)

(21) Appl. No.: 13/156,704

(22) Filed: Jun. 9, 2011

Related U.S. Application Data
(60) Provisional application No. 61/353,005, filed on Jun. 9, 2010.

(51) Int. Cl. A61K 31/555; A61Q 5/00; A61K 31/315; A61P 43/00; A61K 33/04; A61K 31/60

(52) U.S. Cl. 424/702; 424/705; 514/159; 514/186; 514/494

(57) ABSTRACT

The present invention is directed to a stabilized anti-dandruff composition comprising about 10 wt. % to about 40 wt. % of an insoluble anti-dandruff agent, about 6 wt. % to about 12 wt. % of a stabilizer, less than about 1 wt. % of a preservative, and about 0.5 wt. % to about 1 wt. % of a neutralizer, and water. In an embodiment of the present invention, it is directed to a stabilized anti-dandruff composition comprising about 10 wt. % to about 25 wt. % of a zinc-containing layered material, about 6 wt. % to about 12 wt. % of a stabilizer, less than about 1 wt. % of a preservative, and about 0.5 wt. % to about 1 wt. % of a neutralizer, and water.

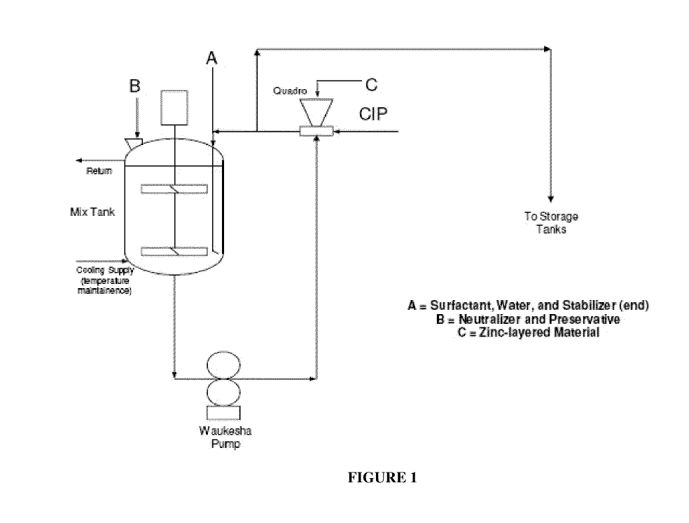

### CROSS REFERENCE TO RELATED APPLICATION

This application claims the benefit of U.S. Provisional Application No. 61/353,005 filed Jun. 9, 2010.

### FIELD OF THE INVENTION

The disclosure generally relates to the preparation of ingredients commonly used in shampoo formulations and, more specifically, to chemically stabilized, insoluble, anti-dandruff and/or antimicrobial compositions that may be incorporated into personal care compositions.

### BACKGROUND OF THE INVENTION

Personal care compositions such as shampoo sometimes contain anti-dandruff agents (or particulates). When present in these compositions, the anti-dandruff agent is typically included in an amount of about 0.01 wt. % to about 5 wt. %, based on the total weight of the personal care composition. In these compositions, the anti-dandruff particulate should be physically- and chemically-compatible with other ingredients of the composition, and should not otherwise unduly impair product stability, aesthetics, or performance.

Anti-dandruff agents suitable for use in personal care compositions include pyridinethione salts, azoles (e.g., ketoconazole, econazole, and elubiol), selenium sulfide, particulate sulfur, salicylic acid, and mixtures thereof. A typical anti-dandruff agent is pyridinethione salt. Personal care compositions can also include a zinc-containing layered material. An example of a zinc-containing layered material can include zinc carbonate materials. Of these, zinc carbonate and pyridinethione salts (particularly zinc pyridinethione or “ZPT) are common in the composition, and often present together. These and other anti-dandruff agents are described in more detail below.

ZPT is typically introduced as a slurry into a batch process of manufacturing shampoo compositions. The slurry typically contains about 25-50% wt. % ZPT, and less than about 1 wt. % surfactant, the balance being water. The slurry is mechanically agitated, continuously, to maintain stability. In the absence of this agitation, the ZPT can be expected to separate from the water within hours (e.g., four hours). Without the homogeneity in this slurry, ZPT will not be evenly distributed in the shampoo and, consequently, the finished composition will suffer from regulatory performance and quality standpoints.

Zinc carbonate is typically introduced into the batch process of manufacturing shampoo compositions as dry material. Specifically, zinc carbonate is typically introduced as a dry powder into one or more of the anionic detersive surfactants and water. The surfactant and water are thereafter combined with other ingredients to form a finished shampoo. The zinc carbonate is introduced into the anionic detersive surfactants and/or water by way of conventional devices suitable for the introduction of otherwise difficult-to-wet powders into liquids, such as those commercially manufactured by Quadro Engineering (Ontario, Canada).

The manufacture of shampoo compositions employing these anti-dandruff agents is somewhat encumbered by the poor stability of the materials in the absence of mechanical agitation (in the case of ZPT) and difficulty in handling (in the case of zinc carbonate). Further, with respect to zinc carbonate, the material is most often incorporated into the shampoo formulation by mixing with one or more major ingredients of the formulation, namely water and/or anionic detersive surfactant. Once mixed with such ingredients, the ingredients are limited to use in formulations that serve as anti-dandruff, personal care compositions. Due to the difficulty in handling and effectively dispersing a difficult-to-wet powder, no other ingredients of the shampoo composition are equally convenient vehicles for introducing zinc carbonate into the shampoo composition. Consequently, a new batch of the ingredients must be made when a shampoo formulation not requiring anti-dandruff qualities is desired.

In view of the foregoing, it would be desirable to provide anti-dandruff compositions that provide more efficient and reliable stability, more efficient manufacture, and more flexibility in their incorporation into personal care compositions.

### SUMMARY OF THE INVENTION

In an embodiment of the present invention, a stabilized anti-dandruff composition includes about 10 wt. % to about 40 wt. % of an insoluble anti-dandruff agent, about 6 wt. % to about 12 wt. % of a stabilizer, less than about 1 wt. % of a preservative, and about 0.5 wt. % to about 1 wt. % of a neutralizer, and water. In another embodiment of the present invention, a stabilized anti-dandruff composition comprising about 10 wt. % to about 25 wt. % of a zinc-containing layered material, about 6 wt. % to about 12 wt. % of a stabilizer, less than about 1 wt. % of a preservative, and about 0.5 wt. % to about 1 wt. % of a neutralizer, and water.

### BRIEF DESCRIPTION OF THE DRAWING

While the specification concludes with claims particularly pointing out and distinctly claiming the subject matter that is regarded as the present invention, it is believed that the invention will be more fully understood from the following description taken in conjunction with the accompanying drawing FIGURE depicting a process for the production of a dispersion containing zinc-layered materials, water, and a detersive anionic surfactant. The FIGURE may have been simplified by the omission of selected elements for the purpose of more clearly showing other elements. Such omissions of elements in the FIGURE is not necessarily indicative of the presence or absence of particular elements in any of the exemplary embodiments, except as may be explicitly delineated in the corresponding written description. The FIGURE is not necessarily to scale.

### DETAILED DESCRIPTION OF THE INVENTION

It has now been discovered that ZPT, zinc carbonate, and other anti-dandruff agents can be more conveniently and efficiently introduced into not only batch processes, but also into continuous processes for manufacturing shampoo compositions. It has been discovered that ZPT and other anti-dandruff agents can be chemically stabilized (versus mechanically stabilized) at room temperature in greater concentrations than heretofore contemplated. Further, the chemically stabilized material will not undesirably separate from its carrier and, therefore, non-homogeneity is not a significant risk. Similarly, it has been discovered that zinc-layered materials, like zinc carbonate, can be chemically stabilized at greater concentrations (i.e., in reduced amounts of carrier fluid(s)), and introduced into a continuous manufacture of a shampoo compositions. More specifically, zinc-layered materials typically were prepared in tanks where the material was present in a 5% active quantity; however, it may now be prepared in a 30% active quantity.

According to one aspect, a stabilized anti-dandruff composition will include about 15 wt. % to about 22 wt. %, of an insoluble, anti-dandruff agent, about 6 wt. % to about 12 wt. %. of a stabilizer, less than about 1 wt. % of a preservative, and about 0.5 wt. % to about 1 wt. % of a neutralizer, the balance being water. In a further embodiment of the present invention, a stabilized anti-dandruff composition will include about 20 wt. %, of an insoluble, anti-dandruff agent, about 10 wt. %. of a stabilizer, less than about 1 wt. % of a preservative, and about 0.5 wt. % to about 1 wt. % of a neutralizer, the balance being water. These ingredients can be combined at room temperature in an agitated tank. A more detailed description of the anti-dandruff agents and stabilizers is set forth below.

According to another aspect, a stabilized zinc-containing layered material composition will include about 10 wt. % to about 25 wt. %, about 6 wt. % to about 12 wt. % of a stabilizer, less than about 1 wt. % preservatives, and about 0.5 wt. % to about 1 wt. % of a neutralizer, the balance being water or the same anionic detersive surfactant(s) that may make up a finished shampoo composition. In a further embodiment of the present invention, a stabilized zinc-containing layered material composition will include about 12 wt. % to about 20 wt. % zinc-containing layered material, about 10 wt. %, of a stabilizer, less than about 1 wt. % preservatives, and about 0.5 wt. % to about 1 wt. % of a neutralizer, the balance being water or the same anionic detersive surfactant(s) that may make up a finished shampoo composition. In an embodiment of the present invention the zinc-containing layered material is basic zinc carbonate. A more detailed description of the zinc-containing layered material and stabilizers is set forth below.

The stabilized zinc-containing layered material composition can be prepared by mixing the ingredients in an agitated tank. Alternatively, it has been found that the composition may be advantageously prepared according to a process of the type shown in the sole drawing FIGURE. The process generally includes introducing into a mix tank an anionic detersive surfactant(s) and water through a dip tube A. The surfactant and water are agitated in the mix tank at a rate sufficient to ensure good homogenization without causing aeration (e.g., 40 rpm to 50 rpm in a 130 L double impeller baffled tank). Preservatives (e.g., at least about 0.25 wt. % sodium benzoate, and about 5 ppm to about 15 ppm of methylchloroisothiazolinone (KATHON®), or mixtures thereof, based on the total weight of the composition) and acid (e.g., about 0.5 wt. % to about 1.0 wt. % of 6N HCl, based on the total weight of the composition) are added to the mix tank through addition port B and agitated (e.g., 40 rpm to 50 rpm in a 130 L double impeller baffled tank). The contents of the mix tank are pumped through a device suitable for dispersing a powder into a liquid and re-enter the mix tank. This is referred to as a recirculating loop. A portion of the zinc-layered material is added on each pass (e.g., an instantaneous ratio of about 5 wt. % of the zinc-layered material to about 95 wt. % of the carrier fluid) via the powder hopper C and pumped through the mixing device, where it combines with the recirculating contents of the mix tank. Recirculation of the contents of the mix tank continues, with additional zinc-layered material added during each cycle, until all of the zinc-layered material has been incorporated. The number of times that recirculation occurs depends on the initial amount of zinc-layered material that is added to the system. In an embodiment where the instantaneous weight ratio of the zinc-layered material to the carrier fluid is about 5 to about 95, about six cycles are required to reach a 30% slurry. After all of the zinc-layered material has been incorporated, a suspending agent (e.g., about 10 wt. % EGDS crystallized out of a surfactant solution) is added to the mix tank through dip tub A to stabilize the dispersion. The stabilized dispersion is then pumped out of the mix tank and to a storage tank.

The stabilized anti-dandruff composition and stabilized zinc carbonate composition can be combined into a single pre-mix prior to introduction to the other ingredients of the shampoo composition. For a continuous process, however, it may be desirable to maintain each in separate containers to permit the greatest freedom in manufacturing different shampoo compositions without burdensome changeover steps.

I. Anti-Dandruff Agents

a. Pyridinethione Salts

Pyridinethione anti-dandruff particulates, especially 1-hydroxy-2-pyridinethione salts, are suitable particulate anti-dandruff agents. The concentration of pyridinethione anti-dandruff particulate typically ranges from about 0.01 wt. % to about 5 wt. %, based on the total weight of the personal care product, generally from about 0.1 wt. % to about 3 wt. %, commonly from about 0.1 wt. % to about 2 wt. %. Suitable pyridinethione salts include those formed from heavy metals such as zinc, tin, cadmium, magnesium, aluminum and zirconium, generally zinc, typically the zinc salt of 1-hydroxy-2-pyridinethione (known as “zinc pyridinethione” or “ZPT”), commonly 1-hydroxy-2-pyridinethione salts in platelet particle form, wherein the particles have an average size of up to about 20 μm, typically up to about 5 μm., commonly up to about 2.5 μm. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. As noted above, ZPT is a preferred pyridinethione salt.

It is further contemplated that when ZPT is used as an anti-microbial particulate in anti-microbial personal care compositions, that an additional benefit of hair growth or re-growth may be stimulated or regulated, or both, or that hair loss may be reduced or inhibited, or that hair may appear thicker or fuller.

Zinc pyrithione may be made by reacting 1-hydroxy-2-pyridinethione (i.e., pyrithione acid) or a soluble salt thereof with a zinc salt (e.g. zinc sulfate) to form a zinc pyrithione precipitate, as illustrated in U.S. Pat. No. 2,809,971.

b. Anti-Microbial Actives

In addition to the anti-dandruff active, shampoo compositions may also include one or more anti-fungal or anti-microbial actives in addition to the metal pyrithione salt actives. Suitable anti-microbial actives include coal tar, sulfur, charcoal, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Typical anti-microbials include itraconazole, ketoconazole, selenium sulphide and coal tar.

i. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01 wt. % to about 5 wt. %, typically from about 0.1 wt. % to about 3 wt. %, and commonly from about 0.3 wt. % to about 2 wt. %, based on the total weight of the personal care product. Especially common for use herein is ketoconazole.

ii. Selenium Sulfide

Selenium sulfide is a particulate anti-dandruff agent suitable for use in anti-microbial personal care compositions, effective concentrations of which range from about 0.1 wt. % to about 4 wt. %, based on the total weight of the personal care product, typically from about 0.3 wt. % to about 2.5 wt. %, commonly from about 0.5 wt. % to about 1.5 wt. %. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), typically less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

iii. Sulfur

Sulfur may also be used as a particulate anti-microbial/anti-dandruff agent in anti-microbial personal care compositions. Effective concentrations of the particulate sulfur are typically from about 1 wt. % to about 4 wt. %, based on the total weight of the personal care product, typically from about 2 wt. % to about 4 wt. %.

iv. Keratolytic Agents

In some embodiments, the personal care composition can further include one or more keratolytic agents such as salicylic acid.

The personal care composition may also include a combination of anti-microbial actives. Such combinations may include octopirox and zinc pyrithione, pine tar and sulfur combinations, salicylic acid and zinc pyrithione combinations, salicylic acid and elubiol combinations, zinc pyrithione and elubiol combinations, octopirox and climbazole combinations, and salicylic acid and octopirox combinations and mixtures thereof.

II. Zinc-Containing Material, Including Zinc Carbonate

Personal care compositions can include a zinc-containing layered material. Those compositions can include about 0.001 wt. % to about 10 wt. % of the zinc-containing layered material based on the total weight of the personal care composition. In an embodiment of the present invention, a personal care composition can include a zinc-containing layered material from about 0.01 wt. % to about 7 wt. % based on the total weight of the personal care composition. In yet a further embodiment of the present invention, a personal care composition can include a zinc-containing layered material from about 0.1 wt. % to about 5 wt. %, based on the total weight of the personal care composition. Suitable zinc-containing layered materials include those described below, including zinc carbonate materials, which are presently preferred:

Zinc-containing layered structures are those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975) Zinc-containing layered materials (ZLM's) may have zinc incorporated in the layers and/or be components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}.nH₂O and some or all of the divalent ions (M²±) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1-3y)/n}.nH₂O where the two metal ions may be different; if they are the same and represented by zinc, the formula simplifies to [Zn₁₋ₓ(OH)₂]^{2x}+2xA⁻.nH₂O. This latter formula represents (where x=0.4) common materials such as zinc hydroxychloride and zinc hydroxynitrate. These are related to hydrozincite as well wherein a divalent anion replace the monovalent anion. These materials can also be formed in situ in a composition or in or during a production process.

These classes of ZLM's represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice

Anti-dandruff efficacy can be dramatically increased in topical compositions by the combination of an anti-dandruff agent with an effective amount of a zinc-containing layered material, wherein the zinc-containing layered material has a specified zinc lability within a surfactant system. Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum.

Labile zinc is maintained by choice of an effective zinc-containing layered material or formation of an effective zinc-containing layered material in-situ by known methods.

Anti-dandruff efficacy can be dramatically increased in topical compositions by the use of polyvalent metal salts of pyrithione, such as zinc pyrithione, in combination with zinc-containing layered materials. Therefore, personal care compositions can include those containing both anti-dandruff agents and zinc-containing layered materials for topical application to provide improved benefits to the skin and scalp (e.g., improved antidandruff efficacy).

A stable composition for zinc-containing layered material dispersion where the zinc source resides in a particulate form is particularly preferred. It has been shown to be challenging to formulate aqueous systems containing a zinc-containing layered material, due to the zinc-containing layered material's unique physical and chemical properties Zinc-containing layered material may have a high density (approximately 3 g/cm³), and needs to be evenly dispersed throughout the product and so it will not aggregate or settle. Zinc-containing layered material also has a very-reactive surface chemistry as well as the propensity to dissolve in systems with pH values below 6.5. A zinc-containing layered material with a solubility of less than 25% will have a measurable % soluble zinc value below a threshold value determined by the weight percent and molecular weight of the zinc compound. The theoretical threshold value can be calculated by the following equation: 0.25 times wt. % Zn Compound in Composition times moles of Zinc in Compound times 65.39 (MW of Zn)/MW of Zn Compound. The pH may be greater than about 6.5; in a further embodiment, the pH may be in a range from about 6.5 to about 12. In yet a further embodiment of the present invention the pH may be from about 6.8 to about 9.5. In yet another embodiment of the present invention the pH may be from about 6.8 to about 8.5.

A smaller particle size is inversely proportional to relative zinc lability. D(90) is the particle size which corresponds to 90% of the amount of particles are below this size. Zinc-containing layered material may have a particle size distribution wherein 90% of the particles are less than about 50 microns, wherein 90% of the particles are less than about 30 microns, wherein 90% of the particles are less than about 20 microns.

There may be a direct relationship between surface area and relative zinc lability. Increased particle surface area generally increases zinc lability due to kinetic factors. Particulate surface area can be increased by decreasing particle size and/or altering the particle morphology to result in a porous particle or one whose overall shape deviates geometrically from sphericity.

The basic zinc carbonate may have a surface area of greater than about 10 m²/g, greater than about 20 m²/g, or greater than about 30 m²/g.

The stabilized zinc-containing layered material composition may further include a coordinating compound with a Log Zn binding constant in a range sufficient to maintain zinc bioavailability. In an embodiment of the present invention, such a coordinating compound has a Log Zn binding constant less than about 6. In a further embodiment of the present invention, such a coordinating compound has a Log Zn binding constant less than about 5. In yet a further embodiment of the present invention, such a coordinating compound has a Log Zn binding constant less than about 4, and greater than about −0.5. In an embodiment of the present invention, such a coordinating compound is an organic acid, strong mineral acid, or coordinating species. Preferred examples of such coordinating compounds include the following (respective Log Zn Binding Constant indicated in parenthesis): EDTA (16.5), EDDS (13.5), EDDA (11.1), NTA (10.7), Xylenol Orange (10.3), Cysteine (9.1), Cystine (6.7), Aspartic Acid (Aspartate) (5.9), Glycine (5.0), Citric Acid (Citrate) (4.8), Glutamic Acid (4.5), Methionine (4.4), Arginine (4.2), Carbonic Acid (Carbonate) (3.9), Ornithine (3.8), Tatronic Acid (Tartrate) (3.2), Malic Acid (Malate) (2.9), Malonic Acid (Malonate) (2.9), Tartaric Acid (Tartrate) (2.7), Adipic Acid (Adipate) (2.6), Phosphoric Acid (Phosphate) (2.4), Phthalic Acid (Phthalate) (2.2), Glycolic Acid (Glycolate) (2.0), Lactic Acid (Lactate) (1.9), Succinic Acid (Succinate) (1.8), Acetic Acid (Acetate) (1.0), Sulfuric Acid (Sulfate) (0.9), Boric Acid (Borate) (0.9), Formic Acid (Formate) (0.6), Chloride (−0.3).

In embodiments having a zinc-containing layered material and a pyrithione or polyvalent metal salt of pyrithione, the ratio of zinc-containing layered material to pyrithione or a polyvalent metal salt of pyrithione is from 5:100 to 10:1. In a further embodiment of the present invention embodiments having a zinc-containing layered material and a pyrithione or polyvalent metal salt of pyrithione, the ratio of zinc-containing layered material to pyrithione or a polyvalent metal salt of pyrithione is from about 2:10 to 5:1. In yet a further embodiment having a zinc-containing layered material and a pyrithione or polyvalent metal salt of pyrithione, the ratio of zinc-containing layered material to pyrithione or a polyvalent metal salt of pyrithione is from 1:2 to 3:1.

III. Stabilizers

Suitable stabilizers for use in stabilizing either the anti-dandruff composition or the zinc-containing layered material composition include cross-linked acrylic acid polymers, trihydroxystearin, and ethylene glycol esters of fatty acids. In an embodiment of the present invention, the cross-linked acrylic acid polymers are those with the CTFA name Carbomer. In a another embodiment of the present invention, the ethylene glycol esters of fatty acids have from about 16 to about 22 carbon atoms. In a further embodiment of the present invention, the stabilizers are the ethylene glycol stearates, both mono and distearate, but particularly the distearate containing less than about 7% of the mono stearate.

IV. Preservatives

The preservative can be any preservative that does not react or degrade when exposed to the pH range of the composition intended to be preserved or under the conditions for the composition's manufacture. Nonlimiting examples of preservatives include sodium benzoate, benzyl alcohol, potassium sorbate, disodium ethylene tetraacetate (Na₂EDTA), tetrasodium ethylenediamine tetraacetate (Na₄EDTA), and mixtures thereof. The preservative can be present in any amount that is effective for killing or inhibiting the growth of microorganisms. The amount of preservative is dependent on the specific preservative used. For example, the preservative can include about 0.25 wt. % sodium benzoate, and about 5 ppm to about 15 ppm of methylchloroisothiazolinone (KATHON®), or mixtures thereof, based on the total weight of the composition

V. Neutralizers

Nonlimiting examples of neutralizers include hydrochloric acid, citric acid, aspartic acid, glutamic acid, carbonic acid, tatronic acid, malic acid, malonic acid, tartaric acid, adipic acid, phosphoric acid, phthalic acid, glycolic acid, lactic acid, succinic acid, acetic acid, sulfuric acid, boric acid, formic acid, and mixtures thereof. The acid is present in any amount that results in pH suitable for maintenance and use of the composition as an ingredient in a shampoo formulation. For example, about 0.5 wt. % to about 1.0 wt. % of 6N HCl can be included in the stabilized anti-dandruff composition and also in the stabilized zinc-containing layered material composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stabilized anti-dandruff composition comprising about 10 wt. % to about 40 wt. % of an insoluble anti-dandruff agent, about 6 wt. % to about 12 wt. % of a stabilizer agent, less than about 1 wt. % of a preservative, and about 0.5 wt. % to about 1 wt. % of a neutralizer, and water.

2. A stabilized anti-dandruff composition according to claim 1 wherein the anti-dandruff agent is from about 15 wt % to about 22 wt %.

3. A stabilized anti-dandruff composition according to claim 1 wherein the anti-dandruff agent is selected from the group consisting of pyridinethione salts, azoles, selenium sulfide, particulate sulfur, salicylic acid and mixtures thereof.

4. A stabilized anti-dandruff composition according to claim 3 wherein the anti-dandruff agent is zinc pyrithione.

5. A stabilized anti-dandruff composition according to claim 1 wherein the stabilizer agent is selected from the group consisting of cross-linked acrylic acid polymers, trihydroxystearin, ethylene glycol esters of fatty acids and mixtures thereof.

6. A stabilized anti-dandruff composition according to claim 5 wherein the stabilizer agent is ethylene glycol stearates.

7. A stabilized anti-dandruff composition according to claim 1 wherein the preservative is selected from the group consisting of sodium benzoate, benzyl alcohol, potassium sorbate, disodium ethylene tetraacetate, tetrasodium ethylenediamine tetraacetate, methylchloroisothiazolinone and mixtures thereof.

8. A stabilized anti-dandruff composition according to claim 1 wherein the neutralizer is selected from the group consisting of hydrochloric acid, citric acid, aspartic acid, glutamic acid, carbonic acid, tatronic acid, malic acid, malonic acid, tartaric acid, adipic acid, phosphoric acid, phthalic acid, glycolic acid, lactic acid, succinic acid, acetic acid, sulfuric acid, boric acid, formic acid, and mixtures thereof.

9. A stabilized anti-dandruff composition comprising about 10 wt. % to about 25 wt. % of a zinc-containing layered material, about 6 wt. % to about 12 wt. % of a stabilizer agent, less than about 1 wt. % of a preservative, and about 0.5 wt. % to about 1 wt. % of a neutralizer, and water.

10. A stabilized anti-dandruff composition according to claim 9 wherein the zinc-containing layered material is from about 12 wt. % to about 20 wt. %.

11. A stabilized anti-dandruff composition according to claim 9 wherein the zinc-containing layered material is basic zinc carbonate.

12. A stabilized anti-dandruff composition according to claim 9 wherein the stabilizer agent is selected from the group consisting of cross-linked acrylic acid polymers, trihydroxystearin, ethylene glycol esters of fatty acids and mixtures thereof.

13. A stabilized anti-dandruff composition according to claim 12 wherein the stabilizer agent is ethylene glycol stearates.

14. A stabilized anti-dandruff composition according to claim 9 wherein the preservative is selected from the group consisting of sodium benzoate, benzyl alcohol, potassium sorbate, disodium ethylene tetraacetate, tetrasodium ethylenediamine tetraacetate, methylchloroisothiazolinone and mixtures thereof.

15. A stabilized anti-dandruff composition according to claim 9 wherein the neutralizer is selected from the group consisting of hydrochloric acid, citric acid, aspartic acid, glutamic acid, carbonic acid, tatronic acid, malic acid, malonic acid, tartaric acid, adipic acid, phosphoric acid, phthalic acid, glycolic acid, lactic acid, succinic acid, acetic acid, sulfuric acid, boric acid, formic acid, and mixtures thereof.

16. A method of making an anti-dandruff composition comprising, combining:
a) an anionic detersive surfactant(s), water, or a mixture thereof in a mix tank;
b) agitating the anionic detersive surfactant(s), water, or a mixture thereof, in the mix tank at conventional rate to provide homogenization;
c) adding preservatives and an acid(s) to the mix tank and agitating at a conventional rate to provide homogenization;
d) pumping the contents of the mix tank through a device to disperse a portion of a zinc-containing layered material on each pass via a powder hopper;
e) recirculating the contents of the mix tank and add zinc-layered material during each cycle until all of the zinc-layered material is incorporated in the contents of the mix tank;
f) adding a stabilizer agent to the mix tank; and
g) adding a premix made by steps a-f above to other ingredients of a shampoo composition.

17. A method of making an anti-dandruff composition according to claim 16 wherein the device to disperse a portion of a zinc-containing layered material is a recirculating loop.

18. A method of making an anti-dandruff composition comprising, combining:
a) an anionic detersive surfactant(s), water, or a mixture thereof in a mix tank;
b) agitating the anionic detersive surfactant(s), water, or a mixture thereof, in the mix tank at conventional rate to provide homogenization;
c) adding preservatives and an acid(s) to the mix tank and agitating at a conventional rate to provide homogenization;
d) adding a slurry containing ZPT to the mix tank and agitating at a conventional rate to provide homogenization;
e) adding a stabilizer agent to the mix tank; and
f) adding a premix made by steps a-e above to other ingredients of a shampoo composition.
